(19) United States
(12) Patent Application Publication
(10) Pub. No.: US 2008/0009938 A1
(43) Pub. Date: Jan. 10, 2008

(54) Stent with a radiopaque marker and method for making the same

(76) Inventors: Bin Huang, Pleasanton, CA (US); David C. Gale, San Jose, CA (US)

Correspondence Address: SQUIRE, SANDERS & DEMPSEY LLP, 1 MARITIME PLAZA, SUITE 300, SAN FRANCISCO, CA, 94111, US

(21) Appl. No.: 11/483,040

(22) Filed: Jul. 7, 2006

(51) Int. Cl. A61F 2/06

(52) U.S. Cl. 623/1.38

(57) ABSTRACT

A stent includes a marker that has a biodegradable body and a plurality of radiopaque nanoparticles dispersed in the body. The marker is disposed in a hole on a biodegradable structural element of the stent, and the marker and the hole have substantially the same configuration. A method for making a stent includes mixing radiopaque nanoparticles with a biodegradable material to form a stent marker, forming a hole on a structural element of a biodegradable stent for accommodating the stent marker, wherein the stent marker and hole have substantially the same configuration, and disposing the stent marker in the hole.

### BACKGROUND OF THE INVENTION

1. Field of the Invention

This invention relates to a stent with a radiopaque marker and a method for making such a stent.

2. Description of the State of the Art

This invention relates to radially expandable endoprostheses, which are adapted to be implanted in a bodily lumen. An “eridoprosthesis” corresponds to an artificial device that is placed inside the body. A “lumen” refers to a cavity of a tubular organ such as a blood vessel. A stent is an example of such an endoprosthesis. Stents are generally cylindrically shaped devices, which function to hold open and sometimes expand a segment of a blood vessel or other anatomical lumen such as urinary tracts and bile ducts. Stents are often used in the treatment of atherosclerotic stenosis in blood vessels. “Stenosis” refers to a narrowing or constriction of the diameter of a bodily passage or orifice. In such treatments, stents reinforce body vessels and prevent restenosis following angioplasty in the vascular system. “Restenosis” refers to the reoccurrence of stenosis in a blood vessel or heart valve after it has been treated (as by balloon angioplasty, stenting, or valvuloplasty) with apparent success.

The structure of stents is typically composed of scaffolding that includes a pattern or network of interconnecting structural elements (struts). The scaffolding can be formed from wires, tubes, or sheets of material rolled into a cylindrical shape. In addition, a medicated stent may be fabricated by coating the surface of either a metallic or polymeric scaffolding with a polymeric carrier. The polymeric scaffolding may also serve as a carrier of an active agent or drug.

The first step in treatment of a diseased site with a stent is locating a region that may require treatment such as a suspected lesion in a vessel, typically by obtaining an x-ray image of the vessel. To obtain an image, a contrast agent, which contains a radiopaque substance such as iodine is injected into a vessel. “Radiopaque” refers to the ability of a substance to absorb x-rays. The x-ray image depicts the lumen of the vessel from which a physician can identify a potential treatment region. The treatment then involves both delivery and deployment of the stent. “Delivery” refers to introducing and transporting the stent through a bodily lumen to a region in a vessel that requires treatment. “Deployment” corresponds to the expanding of the stent within the lumen at the treatment region. Delivery and deployment of a stent are accomplished by positioning the stent about one end of a catheter, inserting the end of the catheter through the skin into a bodily lumen, advancing the catheter in the bodily lumen to a desired treatment location, expanding the stent at the treatment location, and removing the catheter from the lumen. In the case of a balloon expandable stent, the stent is mounted about a balloon disposed on the catheter. Mounting the stent typically involves compressing or crimping the stent onto the balloon. The stent is then expanded by inflating the balloon. The balloon may then be deflated and the catheter withdrawn. In the case of a self-expanding stent, the stent may be secured to the catheter via a retractable sheath or a sock. When the stent is in a desired bodily location, the sheath may be withdrawn allowing the stent to self-expand.

The stent must be able to simultaneously satisfy a number of mechanical requirements. First, the stent must be capable of withstanding the structural loads, namely radial compressive forces, imposed on the stent as it supports the walls of a vessel lumen. In addition to having adequate radial strength or more accurately, hoop strength, the stent should be longitudinally flexible to allow it to be maneuvered through a tortuous vascular path and to enable it to conform to a deployment site that may not be linear or may be subject to flexure. The material from which the stent is constructed must allow the stent to undergo expansion, which typically requires substantial deformation of localized portions of the stent's structure. Once expanded, the stent must maintain its size and shape throughout its service life despite the various forces that may come to bear thereon, including the cyclic loading induced by the beating heart. Finally, the stent must be biocompatible so as not to trigger any adverse vascular responses.

In addition to meeting the mechanical requirements described above, it is desirable for a stent to be radiopaque, or fluoroscopically visible under x-rays. Accurate stent placement is facilitated by real time visualization of the delivery of a stent. A cardiologist or interventional radiologist can track the delivery catheter through the patient's vasculature and precisely place the stent at the site of a lesion. This is typically accomplished by fluoroscopy or similar x-ray visualization procedures. For a stent to be fluoroscopically visible it must be more absorptive of x-rays than the surrounding tissue. Radiopaque materials in a stent may allow for its direct visualization.

In many treatment applications, the presence of a stent in a body may be necessary for a limited period of time until its intended function of, for example, maintaining vascular patency and/or drug delivery is accomplished. Therefore, stents fabricated from biodegradable, bioabsorbable, and/or bioerodable materials may be configured to meet this additional clinical requirement since they may be designed to completely erode after the clinical need for them has ended. Stents fabricated from biodegradable polymers are particularly promising, in part because they may be designed to completely erode within a desired time frame.

However, a significant shortcoming of biodegradable polymers (and polymers generally composed of carbon, hydrogen, oxygen, and nitrogen) is that they are radiolucent with no radiopacity. Biodegradable polymers tend to have x-ray absorption similar to body tissue.

One way of addressing this problem is to attach radiopaque markers to structural elements of the stent. Currently, a small marker ball, made from a radiopaque material such as gold or platinum, is deposited in a hole on the stent to form a marker. The marker ball is secured in the hole by an interference fit or by at least partial filling of the hole. Generally, the diameter of the marker ball is in the range of 0.18 to 0.23 mm.

However, this method of placing a radiopaque marker on a stent has several disadvantages. For example, since the marker ball is small, it is difficult to manipulate it and to place it in the hole. Also the stent hole may cause unevenness on the stent surface, potentially resulting in thrombus. Additionally, there is the risk of the marker being detached from the stent. A detached marker may cause foreign body inflammation.

### SUMMARY OF THE INVENTION

In accordance with one aspect of the present invention, a stent includes a marker that has a biodegradable body and a plurality of radiopaque nanoparticles dispersed in the body. The marker may be disposed in a hole on a biodegradable structural element of the stent, and the marker and the hole preferably have substantially the same configuration. The biodegradable material of the structural element may be substantially the same as the biodegradable material of the marker body. One or more of the nanoparticles may be made from a biostable or biodegradable radiopaque material.

Each of the hole and marker body may be cylindrical and nave a circular cross-section, and the diameter of the hole preferably is substantially the same as the diameter of the marker body. The length of the hole preferably is also substantially the same as the length of the marker body.

In accordance with another aspect of the present invention, a method for making a stent includes mixing radiopaque nanoparticles with a biodegradable material to form a stent marker, forming a hole on a structural element of a biodegradable stent for accommodating the stent marker, wherein the stent marker and hole preferably have substantially the same configuration, and disposing the stent marker in the hole. More specifically, the radiopaque nanoparticles may be mixed with the biodegradable material to form a rod, and the rod may be cut to form stent markers. The stent marker can be welded to the stent structural element by application of heat or by lasing, or can be adhered to the stent structural element by an adhesive.

The present invention has several advantages. First, a marker of the present invention can be larger for a given radiopacity because the marker may be a mixture of a biodegradable radiolucent material and radiopaque particles. A larger marker is easier to handle and to place in the hole. Second, the nanoparticles of the marker are small and less likely to cause foreign body inflammation, and can be more easily absorbed by bodily fluids such as blood without negative impact to bodily functions. Third, since the stent hole and the marker can have substantially the same configuration, the gaps between the stent hole and the marker are small and tend not to cause surface irregularities. Surface irregularities may cause thrombus. Fourth, since the marker body and the stent can be made from the same or similar radiolucent materials, the marker can be more securely attached to the stent.

### BRIEF DESCRIPTION OF THE DRAWINGS

FIG. 1 shows a perspective view of a stent.

FIG. 2 shows stent structural elements with holes for stent makers.

FIG. 3 shows additional stent structural elements with holes for stent makers.

FIG. 4 shows a cylindrical stent marker and a cylindrical hole in which the cylindrical marker is to be placed.

### DETAILED DESCRIPTION OF THE INVENTION

The present invention may be applied to stents and, more generally, implantable medical devices such as, but is not limited to, self-expandable stents, balloon-expandable stents, stent-grafts, vascular grafts, cerebrospinal fluid shunts, pacemaker leads, closure devices for patent foramen ovale, and synthetic heart valves.

A stent can have virtually any structural pattern that is compatible with a bodily lumen in which it is implanted. Typically, a stent is composed of a pattern or network of circumferential and longitudinally extending interconnecting structural elements (struts). In general, the structural elements are arranged in patterns, which are designed to contact the lumen walls of a vessel and to maintain vascular patency. A myriad of structural element patterns are known in the art for achieving particular design goals. A few of the more important design characteristics of stents are radial or hoop strength, expansion ratio or coverage area, and longitudinal flexibility. The present invention is applicable to virtually any stent design and is, therefore, not limited to any particular stent design or pattern. One embodiment of a stent pattern may include cylindrical rings composed of structural elements. The cylindrical rings may be connected by connecting structural elements.

In some embodiments, a stent of the present invention may be formed from a tube by laser cutting the pattern of structural elements in the tube. The stent may also be formed by laser cutting a polymeric or metallic sheet, rolling the pattern into the shape of the cylindrical stent, and providing a longitudinal weld to form the stent. Other methods of forming stents are well known and include chemically etching a polymeric or metallic sheet and rolling and then welding it to form the stent. A polymeric or metallic wire may also be coiled to form the stent. The stent may be formed by injection molding of a thermoplastic or reaction injection molding of a thermoset polymeric material. Filaments of the compounded polymer may be extruded or melt spun. These filaments can then be cut, formed into ring elements, welded closed, corrugated to form crowns, and then the crowns welded together by heat or solvent to form the stent. Lastly, hoops or rings may be cut from tubing stock, the tube elements stamped to form crowns, and the crowns connected by welding or laser fusion to form the stent.

A stent may include a biodegradable material, such as a biodegradable polymer or metal. The biodegradable material may be a pure or substantially pure biodegradable polymer or metal. Alternatively, the biodegradable material may be a mixture of at least two types of biodegradable polymers and metals.

In some embodiments, the composition of the stent may be modified or tuned to obtain a desired erosion rate. For example, the erosion rate of the stent may be increased by increasing the fraction of a faster eroding component in the stent material. As indicated above, a stent made from a biodegradable polymer or metal is intended to remain in the body for a duration of time until its intended function of, for example, maintaining vascular patency and/or drug delivery is accomplished. After the process of degradation, erosion, absorption, and/or resorption has been completed, no portion of the biodegradable stent, or a biodegradable portion of the stent will remain. In some embodiments, very negligible traces or residue may be left behind. The duration can be in a range from about a month to a few years. However, the duration is typically in a range from about one month to twelve months, or in some embodiments, six to twelve months. It is important for the stent to provide mechanical support to a vessel for at least a portion of the duration. Many biodegradable polymers have erosion rates that make them suitable for treatments that require the presence of a device in a vessel for the above-mentioned time-frames.

Representative examples of biosoluble materials that may be used to fabricate embodiments of stents include, but are not limited to, poly(ethylene oxide); poly(acrylamide); poly(vinyl alcohol); cellulose acetate; blends of biosoluble polymer with bioabsorbable and/or biostable polymers; N-(2-hydroxypropyl)methacrylamide; and ceramic matrix composites.

Representative examples of biodegradable metals that may be used to fabricate a stent may include, but are not limited to, magnesium, zinc, and iron. Representative mixtures or alloys may include magnesium/zinc, magnesium/iron, zinc/iron, and magnesium/zinc/iron. In some embodiments, biodegradable metals erode as a result of a chemical reaction between a metal surface and its environment. Erosion or corrosion in a wet environment, such as a vascular environment, results in removal of metal atoms from the metal surface. The metal atoms at the surface lose electrons and become charged ions that leave the metal to form salts in solution. Preferably, the biodegradable metals are biocompatible so that they do not negatively impact bodily functions.

Biodegradation refers generally to changes in physical and chemical properties that occur in a material upon exposure to bodily fluids as in a vascular environment. The changes in properties may include a decrease in molecular weight, deterioration of mechanical properties, and decrease in mass due to erosion or absorption. Mechanical properties may correspond to strength and modulus of the material. Deterioration of the mechanical properties of the material decreases the ability of a stent, for example, to provide mechanical support in a vessel. The decrease in molecular weight may be caused by, for example, hydrolysis, oxidation, enzymolysis, and/or metabolic processes.

The terms “biodegradable,” “bioabsorbable,” and “bioerodable,” as well as the terms “degraded,” “eroded,” and “absorbed,” are used interchangeably and refer to materials that are capable of being completely eroded or absorbed when exposed to bodily fluids such as blood and can be gradually resorbed, absorbed and/or eliminated by the body.

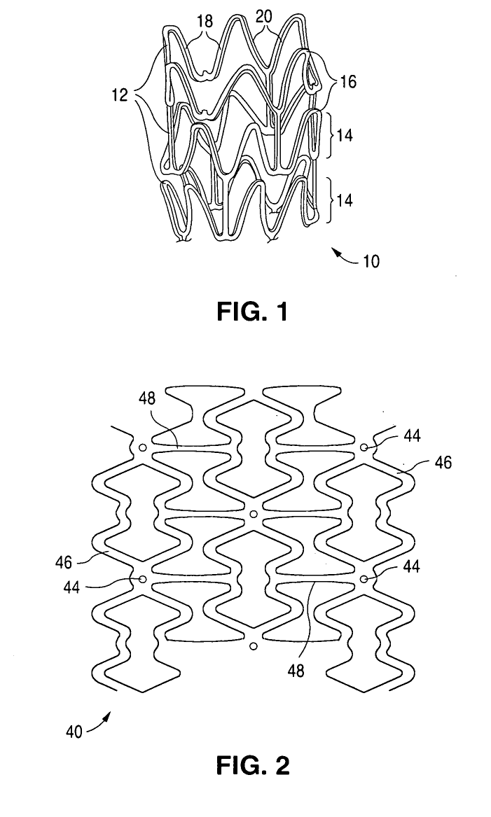

FIG. 1 illustrates a three-dimensional view of a cylindrically-shaped stent 10 with structural elements 12 that form cylindrical rings 14 that are connected by linking structural elements 12. The cross-section of the structural elements 12 in the stent 10 is rectangular-shaped. The structural elements 12 have abluminal faces 16, luminal faces 18, and sidewall faces 20. The cross-section of structural elements is not limited to what has been illustrated, and therefore, other cross-sectional shapes are applicable with embodiments of the present invention. The pattern should not be limited to what has been illustrated as other stent patterns are easily applicable with embodiments of the present invention.

Various embodiments of the present invention include a radiolucent stent with one or more radiopaque markers disposed within one or more holes on the stent. A hole may be located at any suitable location on the stent. For example, the hole may be located on a stent element or at an intersection of three or more stent elements. And the hole can be on an abluminal, luminal or side surface of the stent. Preferably, the holes are distributed in a manner that facilitates visualization of the stent during and after implantation. In some embodiments, it may be advantageous to limit the placement of holes to particular locations or portions of surfaces of a stent. For example, to delineate just the margins of the stent so that the physician may see its full length, markers can be placed only at the stent ends. In some other embodiments, the markers may be distributed circumferentially and longitudinally throughout a stent.

FIG. 2 depicts one embodiment of a stent pattern 40 with holes 44 for receiving markers. In FIG. 2, the stent pattern 40 is shown in a flattened condition showing an abluminal or luminal surface so that the pattern can be clearly viewed. When the flattened portion of the stent pattern 40 is in a cylindrical condition, it forms a radially expandable stent. Stent pattern 40 includes cylindrically aligned structural elements 46 and linking structural elements 48. In this embodiment, the holes 44, which may be blind or through holes, are located at an intersection of six structural elements or of five structural elements. The term “blind hole” as used herein designates a hole that has one open end and one closed end and does not pass completely through a stent element, and the term “through hole” as used herein designates a hole that has two open ends and passes completely through a stent element.

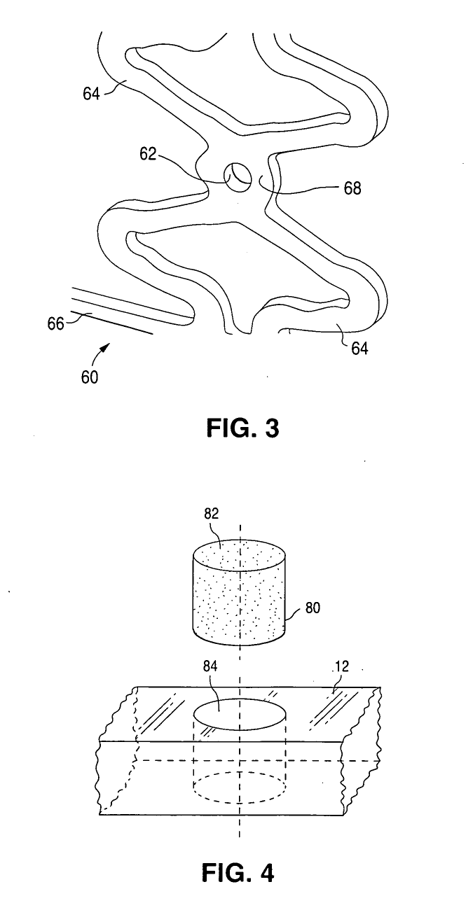

FIG. 3 depicts a three-dimensional view of another stent pattern 60 with holes 62. The stent pattern 60 includes cylindrically aligned structural elements 64 and linking structural elements 66. A hole 62 is located in an intersection 68 of four structural elements. The hole 62 has a cylindrical shape and passes completely through the radial thickness of the intersection 68.

Although the holes illustrated in FIGS. 2 and 3 have a cylindrical configuration with a circular cross-section. A hole may have any suitable configuration. For example, a hole may have a cylindrical configuration with a polygonal or elliptical cross-section.

As shown in FIG. 4, each marker 80 (with nanoparticles 82) and the corresponding hole 84, in which the marker 80 is disposed, preferably have substantially the same configuration. For example, if the hole 84 has a cylindrical configuration, the marker 80 preferably also has a cylindrical configuration. Further, the marker's cross-section may be substantially the same as the hole's cross-section. For example, if the hole 82 has a circular cross-section, the marker 80 may also have a circular cross-section, and the marker's diameter may be within 80% to 100%, 90% to 100%, 95% to 100%, 98% to 100%, or 99% to 100% of the hole's diameter, or may be the same as the hole's diameter. Similarly, the marker's length may be substantially the same as the hole's length. For example, the marker's length may be within 80% to 120%, 90% to 110%, 95% to 105%, 98% to 102%, or 99% to 110% of the hole's length, or may be the same as the hole's length.

Each marker may include a biodegradable material and a plurality of radiopaque particles dispersed in the biodegradable material. In certain embodiments, the radiopaque particles may be nanoparticles. A “nanoparticle” refers to a particle with a dimension in the range of about 1 nm to about 1000 nm, preferably of about 10 nm to about 900 nm, more preferably of about 200 nm to about 800 nm, most preferably of about 400 nm to about 600 nm. A significant advantage of nanoparticles over larger particles is that nanoparticles may disperse more uniformly in a polymeric matrix, which results in more uniform properties such as radiopacity and erosion rate. Additionally, nanoparticles may be more easily absorbed by bodily fluids such as blood without negative impact to bodily functions.

The biodegradable material of a stent marker may be any of the biodegradable stent materials described above. The biodegradable material of a stent marker preferably is the same as or similar to the biodegradable material of the stent. The stent marker may degrade at the same or substantially the same rate as the stent. For instance, the marker may be configured to completely or almost completely degrade at the same time or approximately the same time as the stent. In other embodiments, the marker may degrade at a faster rate than the stent. In this case, the marker may completely or almost completely degrade before the stent is completely degraded. In still other embodiments, the marker may degrade at a slower rate than the stent.

The radiopaque particles of a stent marker may be composed of one or more biostable or biodegradable radiopaque materials. The biostable materials may be biostable metals. Representative examples of biostable metals include, but are not limited to, platinum and gold. The biodegradable materials of the radiopaque particles may be biodegradable metals and/or metallic compounds such as biodegradable metallic oxides, biocompatible metallic salts, gadolinium salts, and iodinated contrast agents. In one embodiment, the radiopaque particles may be composed of a pure or substantially pure biodegradable metal. Alternatively, the radiopaque particles may be a mixture or alloy of at least two types of metals. Representative examples of biodegradable metals may include, but are not limited to, magnesium, zinc, and iron. Representative mixtures or alloys may include magnesium/zinc, magnesium/iron, zinc/iron, and magnesium/zinc/iron. Representative radiopaque compounds may be iodine salts, bismuth salts, or barium salts.

The radiopacity of the marker can be adjusted by varying the number of radiopaque particles in the marker or by varying the radiopacity of the particles. In some embodiments, the radiopaque particles may be between 10% and 80%; 20% and 70%; 30% and 60%; or 40% and 50% of the marker by volume. The radiopacity of the marker can be increased by using a more radiopaque metal to make the particles or to make a higher portion of the particles.

The manufacturing of a stent of the present invention involves some or all of the following steps. A stent is manufactured in a know manner, preferably using a biodegradable material. Then the appropriate configurations, number and locations of stent markers are determined, and holes for accommodating the markers are made on the stent using, for example, laser. Each hole may be a through hole or a blind hole. Radiopaque particles, preferably radiopaque nanoparticles, are mixed with a biodegradable material. The mixture is then molded or extruded into cylindrical rods. The cylindrical rods are cut into cylindrical markers. Preferably, the length of each marker is substantially the same as that of the hole, in which the marker is to be disposed. The markers are then disposed in the respective holes.

Each marker can be secured in the corresponding hole in any suitable manner. For example, the marker can be welded to the stent by an application of heat or by lasing. In certain embodiments, the marker may additionally or alternatively be coupled within the hole with any suitable biocompatible adhesive. In one embodiment, the adhesive may include a solvent. The solvent may dissolve the polymer of the structural element within the hole to allow the marker within the hole to be coupled to the structural element. For markers that include a polymer, a solvent may also dissolve a portion of the marker. In another embodiment, the adhesive may include a solvent mixed with a polymer. The solvent or the solvent-polymer mixture may be applied to the structural element within the hole or the marker followed by disposing the marker within the hole. The solvent may then be removed through evaporation. Evaporation may be facilitated by, for example, heating the structural element in an oven or by some other method.

Representative examples of solvents may include, but are not limited to, chloroform, acetone, chlorobenzene, ethyl acetate, 1,4-dioxane, ethylene dichloride, 2-ethyhexanol, and combinations thereof. Representative polymers may include biostable and biodegradable polymers disclosed herein that may be dissolved by the selected solvent. In other embodiments, adhesives may include, but are not limited to, thermosets such as, for example, epoxies, polyesters and phenolics; thermoplastics such as, for example, polyamides, polyesters and ethyl vinyl acetate (EVA) copolymers; and elastomers such as, for example, natural rubber, styrene-isoprene-styrene block copolymers, and polyisobutylene. Other adhesives include, but are not limited to, proteins; cellulose; starch; poly(ethylene glycol); fibrin glue; and derivatives and combinations thereof.

When a marker is disposed in a hole, there may be gaps between the marker and the internal surface of the hole. Such gaps may interfere with the structure of a lumen and/or with flow of bodily fluid through the lumen and may result in formation of turbulent and stagnant zones which can act as a nidus for thrombosis. In the present invention, since the marker has substantially the same configuration as the hole, the gaps between the marker and the hole are small and may be eliminated by the welding or adhesion of the marker to the hole. However, if small gaps remain, a stent coating material may be applied to fill the gaps. The coating material may be applied in various ways known in the art such as by spraying or dipping.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

1. A stent comprising:
a biodegradable structural element including a hole; and
a marker disposed in the hole, the marker including
a biodegradable body having substantially the same configuration as the hole, and
a plurality of radiopaque particles dispersed in the body.

2. The stent of claim 1, wherein each of the hole and marker body is cylindrical and has a circular cross-section.

3. The stent of claim 2, wherein the diameter of the hole is substantially the same as the diameter of the marker body.

4. The stent of claim 1, wherein a length of the hole is substantially the same as a length of the marker body.

5. The stent of claim 1, wherein at least one of the particles comprise a biostable radiopaque material.

6. The stent of claim 1, wherein at least one of the particles comprise a biodegradable radiopaque material.

7. The stent of claim 1, wherein the particles have a dimension in the range of about 1 nm to about 1000 nm.

8. The stent of claim 1, wherein the nanoparticles have a dimension in the range of about 10 nm to about 900 nm.

9. The stent of claim 1, wherein the nanoparticles have a dimension in the range of about 200 nm to about 600 nm.

10. The stent of claim 1, wherein the biodegradable material of the structural element is substantially the same as the biodegradable material of the marker body.

11. A method for making a stent, comprising:
mixing radiopaque particles with a biodegradable material to form a stent marker;
forming a hole on a structural element of a biodegradable stent for accommodating the stent marker, wherein the stent marker and hole have substantially the same configuration; and
disposing the stent marker in the hole.

12. The method of claim 11, wherein the step of mixing includes mixing the radiopaque nanoparticles with the biodegradable material to form a rod and cutting the rod to form the stent marker.

13. The method of claim 11, wherein each of the hole and marker body has a circular cross-section.

14. The method of claim 13, wherein the diameter of the hole is substantially the same as the diameter of the marker body.

15. The method of claim 11, wherein the length of the hole is substantially the same as the length of the marker body.

16. The method of claim 11, wherein at least one of the particles comprise a biostable radiopaque material.

17. The method of claim 11, wherein at least one of the particles comprise a biodegradable radiopaque material.

18. The method of claim 11, wherein the nanoparticles have a dimension in the range of about 1 nm to about 1000 nm.

19. The method of claim 11, wherein the nanoparticles have a dimension in the range of about 10 nm to about 900 nm.

20. The method of claim 11, wherein the nanoparticles have a dimension in the range of about 200 nm to about 600 nm.

21. The method of claim 11, wherein the biodegradable material of the structural element is substantially the same as the biodegradable material of the marker.

22. The method of claim 11, further comprising welding the stent marker to the stent structural element by application of heat.

23. The method of claim 11, further comprising welding the stent marker to the stent structural element by lasing.

24. The method of claim 11, further comprising adhering the stent marker to the stent structural element by application of an adhesive.
